(19) United States
(12) Patent Application Publication
(10) Pub. No.: US 2019/0343873 A1
(43) Pub. Date: Nov. 14, 2019

(54) SEQUENCE-SELECTIVE GENE EXPRESSION REGULATORS

(71) Applicant: KYOTO UNIVERSITY, Kyoto (JP)

(72) Inventors: Hiroshi SUGIYAMA, Kyoto-shi (JP); Kazuo SERIE, Kyoto-shi (JP)

(73) Assignee: KYOTO UNIVERSITY, Kyoto (JP)

(21) Appl. No.: 16/410,046

(22) Filed: May 13, 2019

(30) Foreign Application Priority Data
May 14, 2018 (JP) 2018-093273

(51) Int. Cl. A61K 31/787; A61K 47/54; A61K 47/56; A61K 47/64

(52) U.S. Cl. CPC A61K 31/787; A61K 47/545; A61K 47/64; A61K 47/56; A61K 47/555

(57) ABSTRACT

A conjugate comprising a pyrrole-imidazole polyamide that recognizes a specific DNA sequence and a bromodomain inhibitor, a composition for regulating biochemical activity, specifically for regulating histone modification, e.g., for inducing histone acetylation, which comprises the conjugate, a composition for recruiting a bromodomain-containing protein which comprises the conjugate, a method for nucleosome acetylation which comprises using the conjugate, and a method for bromodomain-containing protein recruitment which comprises using the conjugate are provided.

### TECHNICAL FIELD

The present disclosure is directed to a conjugate including a pyrrole-imidazole polyamide that recognizes a specific DNA sequence and a bromodomain inhibitor (Bi); a composition containing such a conjugate for use in regulating biochemical activity, specifically histone modification such as histone acetylation; a composition containing the conjugate for use in recruiting a bromodomain (BD)-containing protein; a method for nucleosome acetylation utilizing the conjugate; or a method for BD-containing protein recruitment utilizing the conjugate.

### BACKGROUND OF THE INVENTION

Eukaryotic genomes have their DNAs packed in form of chromatin into the nucleus. The chromatin has nucleosomes as the fundamental repeating units. Each of the units consists of a segment of DNA and histones. It is believed that the structure of chromatin and gene expression are regulated by epigenetic modifications of histones and DNA.

Posttranslational modifications (PTMs) on histone proteins play significant roles in epigenetic regulation of eukaryotic chromatin. Acetylation of lysine residues is one of the major PTMs on histones, and is strongly correlated with transcriptional activation. The code governing histone acetylation is regulated by histone acetyltransferases (HATs) as writers and by histone deacetylases (HDACs) as erasers. Acetylated histones are generally found in euchromatin and activated gene regions, and known to operate via two mechanisms. One is to decrease the positive charge of histone proteins, resulting in weakened interaction between histones and DNA, which in turn open up the chromatin structure. The other is mediated by bromodomain (BD)-containing proteins, which selectively bind to acetylated lysine residues. Although locus-specific (i.e., DNA sequence-specific) regulation of histone acetylation has been shown to be essential in biological processes, techniques for controlling histone acetylation at any region of interest remain limited.

Pyrrole-imidazole polyamides (hereinafter, referred to as “PI polyamides” or “PIP”) which consist of N-methyl pyrrole and N-methyl imidazole are synthetic oligomers that recognize specific DNA sequences located within the minor groove by virtue of their pyrrole (P) and imidazole (I) pairs interlocked by a hairpin linkage. In the anti-parallel structure, a pair of pyrrole and imidazole (P/I) recognizes C⋅G base pair, a pair of pyrrole and pyrrole (P/P) recognizes A⋅T or T⋅A base pair, and a pair of imidazole and pyrrole (I/P) recognizes G⋅C base pair. PI polyamides can specifically bind to any double-stranded DNA sequence by virtue of the above recognitions. Thus, designing the order of PI pairs enables in vivo delivery of PI polyamides to the targeted site in genome. PI polyamides targeting nuclear DNAs have been variously applied.

In recent study, PI polyamides were conjugated with a potent histone deacetylase (HDAC) inhibitor, suberoylanilide hydroxamic acid-conjugated (SAHA) (see Pandian, G. N. et al., Sci Rep 4, 3843, doi:10.1038/srep03843 (2014); and Saha, A. et al., Bioorg Med Chem 21, 4201-4209, doi:10.1016/j. bmc. 2013. 05. 002 (2013)). PI polyamides were also conjugated with a HAT-activating compound, N-(4-chloro-3-(trifluoromethyl)phenyl)-2-ethoxybenzamide-conjugated (CTB) (see WO2016/129680). The SAHA-conjugated PI polyamides and the CTB-conjugated PI polyamides were shown to specifically upregulate the expression of a targeted gene. However, the level of gene expression activation is not stable.

### SUMMARY OF THE INVENTION

An object of the present disclosure is to provide techniques for introducing specific epigenetic modifications into specific DNA regions or specific nucleosomes.

It is believed that HATs having BD bind to acetyl lysine via BD and acetylate lysine near HAT domain, thus acetylation is propagated. The present inventors paid their attention to such a natural propagation system of acetylation. As a result of intensive study, the present inventors succeeded in development of techniques for introducing modifications by BD-containing proteins into specific regions on DNAs, which comprises DNA sequence-selectively recruiting the BD-containing proteins by utilizing BD inhibitors that bind to BD and PI polyamides that sequence-selectively bind to DNAs. Specifically, the present inventors conjugated a BD inhibitor (hereinafter also referred to as “Bi”) and a PI polyamide to generate a Bi-PI polyamide conjugate (hereinafter also referred to as “Bi-PIP”), and thereby completed a series of novel techniques.

The present disclosure provides the following aspects:

(1) a conjugate comprising a pyrrole-imidazole polyamide that recognizes a specific DNA sequence and a bromodomain inhibitor,

(2) the conjugate according to (1), wherein the bromodomain inhibitor is a compound that interacts with a bromodomain contained in a protein selected from the group consisting of a histone acetyltransferase, a histone methyltransferase, an ATP-dependent chromatin-remodeling complex protein, and a BET protein,

(3) the conjugate according to (2), wherein the bromodomain inhibitor is a compound that interacts with a histone acetyltransferase,

(4) the conjugate according to (1), wherein the bromodomain inhibitor is a 5-isoxazolyl-benzimidazole compound,

(5) the conjugate according to (4), wherein the 5-isoxazolyl-benzimidazole compound is a compound represented by the following formula:

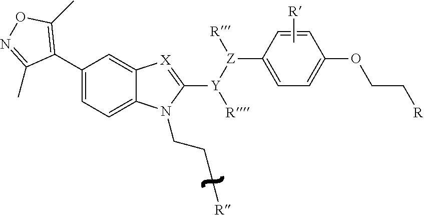

wherein,

R is NH₂ or NHCOCH₂CH₂CH₂COOH,

R′ is H, CH₃, F, Cl, or NO₂,

R″ is a group represented by the following formula:

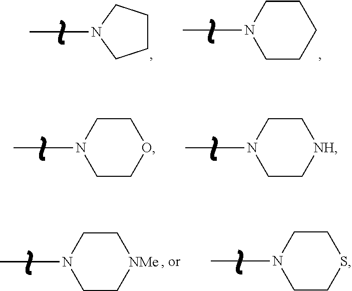

R′″ is H or CH₃,

R″″ is H or CH₃,

X is CH or N,

Y is CH or O, and

Z is CH or O,

(6) the conjugate according to (3), which is represented by Formula I:

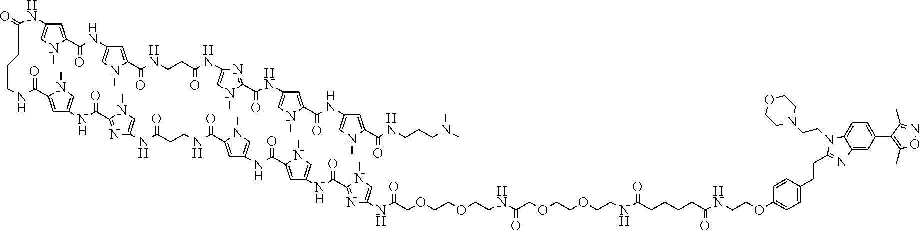 [SMILES: Cc1noc(C)c1-c1ccc2c(c1)nc(CCc1ccc(OCCNC(=O)CCCCC(=O)NCCOCCOCC(=O)NCCOCCOCC(=O)Nc3cn(C)c(C(=O)Nc4cc(C(=O)Nc5cc(C(=O)NCCC(=O)Nc6cn(C)c(C(=O)Nc7cc(C(=O)NCCCC(=O)Nc8cc(C(=O)Nc9cc(C(=O)NCCC(=O)Nc%10cn(C)c(C(=O)Nc%11cc(C(=O)Nc%12cc(C(=O)NCCCN(C)C)n(C)c%12)n(C)c%11)n%10)n(C)c9)n(C)c8)n(C)c7)n6)n(C)c5)n(C)c4)n3)cc1)n2CCN1CCOCC1]

### or Formula II:

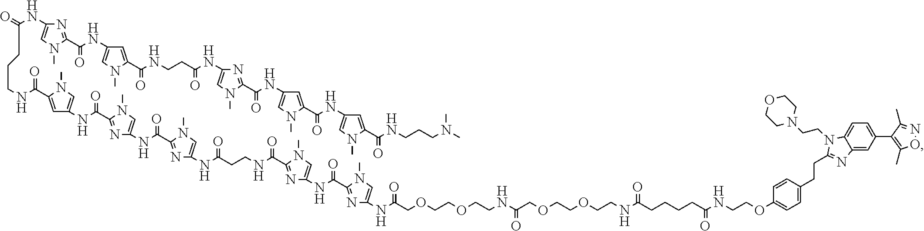 [SMILES: Cc1noc(C)c1-c1ccc2c(c1)nc(CCc1ccc(OCCNC(=O)CCCCC(=O)NCCOCCOCC(=O)NCCOCCOCC(=O)Nc3cn(C)c(C(=O)Nc4cn(C)c(C(=O)NCCC(=O)Nc5cn(C)c(C(=O)Nc6cn(C)c(C(=O)Nc7cc(C(=O)NCCCC(=O)Nc8cn(C)c(C(=O)Nc9cc(C(=O)NCCC(=O)Nc%10cn(C)c(C(=O)Nc%11cc(C(=O)Nc%12cc(C(=O)NCCCN(C)C)n(C)c%12)n(C)c%11)n%10)n(C)c9)n8)n(C)c7)n6)n5)n4)n3)cc1)n2CCN1CCOCC1]

(7) a composition for regulating biochemical activity, comprising the conjugate according to any one of (1) to (6),

(8) a composition for regulating modification of histone, comprising the conjugate according to any one of (1) to (6),

(9) a method of DNA sequence-selectively acetylating a nucleosome, the method comprising bringing the conjugate according to any one of (3) to (6) into contact with a sample containing a nucleosome,

(10) a composition for recruiting a bromodomain-containing protein, comprising the conjugate according to any one of (1) to (6), and

(11) a method of DNA sequence-selectively recruiting a bromodomain-containing protein, the method comprising bringing the conjugate according to any one of (1) to (6) into contact with a sample.

According to the present disclosure, as an example, a desired modification is introduced to a specific nucleosome containing a target DNA sequence by utilizing Bi-PIP which is a conjugate of a PI polyamide that recognizes the target DNA sequence with an inhibitor against BD of a desired BD-containing protein. In an aspect of the present disclosure, Bi-PIP selectively binds to a target DNA sequence on a nucleosome and recruits a desired BD-containing protein (e.g. HAT) to the vicinity of the target sequence via Bi, and thereby a desired modification (e.g. acetylation of a lysine residue) is introduced near the target sequence. For example, the technique for modification of target nucleosomes by Bi-PIP as disclosed herein can be applied to elucidation of a mechanism of epigenetic modification crosstalk in nucleosome units, epigenome editing, expression regulation of endogenous genes, etc.

### BRIEF DESCRIPTION OF THE DRAWINGS

FIG. 1 shows chemical structures of Bi-PIP, Bi and PIP used in Examples. In FIG. 1, Me represents a methyl group.

FIG. 2A is a schematic view showing DNA templates used in Examples.

FIG. 2B is a schematic view showing HAT reaction-in vitro ChIP-qPCR performed in Examples.

FIG. 3 is a graph showing results of acetylation of a target nucleosome induced by Bi-PIP. HAT reaction-in vitro ChIP-qPCR was performed with a series of P300 concentrations with or without 100 nM Bi-PIP1. Positive control represents a nucleosome with synthetic acetylated histone H3 (K4ac, K9ac, K14ac, K18ac, K23ac). The figure shows, from left to right, results of, positive control, P300 (0, 1, 3, 10, 30, and 100 nM) plus vehicle group, and P300 (0, 1, 3, 10, 30, and 100 nM) plus Bi-PIP1 group.

FIG. 4 is a graph showing results of acetylation of a target nucleosome induced by Bi-PIP1, PIP1 monomer, Bi monomer, and a mixture of Bi and PIP1. HAT reaction-in vitro ChIP-qPCR was performed with 10 nM of P300. Each compound was applied at a concentration of 100 nM.

FIG. 5 is a graph showing results of acetylation of a target nucleosome induced by Bi-PIP1 (100 nM) and Bi-PIP2 (100 nM). HAT reaction-in vitro ChIP-qPCR was performed with 10 nM of P300.

FIG. 6 is a graph showing results of acetylation of a target nucleosome in a nuclear extract induced by Bi-PIP.

FIG. 7A shows selective gene activation in cultured cells by Bi-PIP. Volcano plots of transcriptome comparison of Bi-PIP1 vs vehicle (left) and Bi-PIP2 vs vehicle (right) are shown.

FIG. 7B is a graph showing selective gene activation in cultured cells by Bi-PIP. Expression of NTS and IER5L in cultured cells in the presence of Bi-PIP1 or Bi-PIP2 was confirmed by RT-qPCR. Error bars represent standard deviation of data obtained from two culture wells.

### DETAILED DESCRIPTION OF THE EMBODIMENTS

### 1. PI Polyamides

PI polyamides are generally polyamides containing N-methylpyrrole units (P), N-methylimidazole units (I) and a γ-aminobutyric acid moiety, in which P, I and the γ-aminobutyric acid moiety are linked to one another via amide bonds (—C(═O)—NH—) (Trauger et al, Nature, 382, 559-61(1996); White et al, Chem. Biol., 4, 569-78(1997); and Dervan, Bioorg. Med. Chem., 9, 2215-35 (2001)). The PI polyamides are wholly folded in a U-shaped conformation (hairpin form) by the γ-aminobutyric acid moiety serving as a linker (γ-linker). In the U-shaped conformation, two chains containing P and I are arranged in parallel, flanking the linker. When pairs containing P and I formed between the two chains are specific combinations of P and I (P/I pair, I/P pair, or P/P pair), they can bind to specific base pairs in DNA with high affinity. For example, P/I pair can bind to C⋅G base pair and I/P pair can bind to G⋅C base pair. For example, P/P pair can bind to both A⋅T base pair and T⋅A base pair. The PI polyamides may contain 3-hydroxypyrrole (Hp) or β-alanine in addition to P and I. P may be replaced with Hp or β-alanine. For example, Hp/P pair can bind to T⋅A base pair (White et al., Nature, 391, 468-71 (1998)). For example, β-alanine/β-alanine pair can bind to T⋅A base pair and A⋅T base pair. For example, β-alanine/I pair can bind to G⋅C base pair, and I/β-alanine pair can bind to G⋅C base pair. For example, β-alanine/P pair and P/β-alanine pair can bind to both of T⋅A base pair and A⋅T base pair. In addition, the γ-linker moiety, and β-alanine attached to the N-terminus of PI polyamides can bind to both of T⋅A base pair and A⋅T base pair. The PI polyamides that recognize and bind to a target DNA sequence can be designed by changing the paring combinations of P, I, Hp and/or β-alanine according to the DNA sequence of the target.

As used herein, in the PI polyamides, a methyl group on a nitrogen atom at position 1 of P or I may be substituted by hydrogen or an alkyl group other than a methyl group. Examples of the alkyl group other than a methyl group include a C2-C10 linear, branched, or cyclic saturated or unsaturated alkyl group, preferably a C2-C5 linear, branched, or cyclic saturated or unsaturated alkyl group, and for example, ethyl, n-propyl, isopropyl, n-butyl, sec-butyl, isobutyl, tert-butyl and the like are included. The alkyl group and the methyl group may be substituted. For example, methylene in the alkyl group may be substituted by oxygen or the like. Further, as used herein, hydrogen at position 3 of P in the PI polyamides may be substituted by a hydroxyl group. As used herein, terms “P” or “pyrrole unit” and “I” or “imidazole unit” include an N-substituted pyrrole unit, an N-unsubstituted pyrrole unit, a 3-hydroxypyrrole unit, an N-substituted imidazole unit, and an N-unsubstituted imidazole unit.

As used herein, the γ-linker may have a side chain at its α-position or β-position. For example, the γ-linker may have an amino group at its α-position or β-position. In other words, the γ-linker may be a linker consisting of an N-α-N-γ-diaminobutyric acid residue or an N-β-N-γ-diaminobutyric acid residue. In addition, the side chain may be modified with a molecule such as a fluorescent group or biotin.

As used herein, the PI polyamides may be modified at the N terminus and/or C terminus with various functional groups or molecules. The functional groups or molecules to be attached to the N terminus and/or C terminus of the PI polyamides can be determined as appropriate by a person skilled in the art. For example, various functional groups can be attached to the N terminus and/or C terminus via an amide linkage. Examples of the functional groups include, but not limited to, a carboxyl group such as a β-alanine residue or a γ-aminobutyric acid residue, an acetyl group, and an amino group. For example, an acetyl group may be attached to the N-terminus of the PI polyamides. For example, a dimethylaminopropylamino group may be attached to the C-terminus of the PI polyamides. The N terminus and/or C terminus of the PI polyamides may be modified with a molecule such as a fluorescent group, biotin, or isophthalic acid. As used herein, examples of the fluorescent group include, but not limited to, fluorescein, rhodamine dyes, cyanine dyes, ATTO dyes, Alexa Fluor dyes, and BODIPY. The fluorescein includes fluorescein derivatives (for example, fluorescein isothiocyanate).

As used herein, the PI polyamides may be modified to maintain or improve the ability to bind to DNA. Examples of the modified PI polyamides include, but not limited to, PI polyamides containing an amino group attached to position α or β of the γ-linker (i.e., PI polyamides having a γ-linker consisting of an N-α-N-γ-diaminobutyric acid residue or an N-β-N-γ-diaminobutyric acid residue), PI polyamides containing an amino group attached to position α or β of the γ-linker in which the amino group is further modified with a molecule such as a fluorescent group or biotin, PI polyamides modified with a molecule such as a fluorescent group or biotin at the N terminus, and PI polyamides modified with a molecule such as isophthalic acid at the C terminus.

As used herein, the PI polyamides are designed to recognize a specific DNA sequence, that is, any desired DNA sequence. For example, the desired DNA sequence may be a part of the sequence of a gene, or its related sequence, which is desired to be modified or of which the expression is desired to be regulated. Regulation of expression includes activation and suppression of expression.

As used herein, the number of pyrrole-imidazole pairs constituting the PI polyamide which are formed by P, I, Hp and/or β-alanine is not limited as long as it is at least 2. For example 3 to 12, preferably 4 to 10, more preferably 4 to 8 pyrrole-imidazole pairs constitute the PI polyamide. When the PI polyamide is designed to have a DNA sequence-recognition part consisting of at least 5 pyrrole-imidazole pairs, it is preferably designed to contain β-alanine. As used herein, the term “pyrrole-imidazole pair(s)” includes any pairs of P, I, Hp and β-alanine.

Methods for designing and producing the PI polyamides are known (see, e.g., JP-B 3045706, JP-A 2001-136974, WO03/000683, JP-A 2013-234135, and JP-A 2014-173032). For example, the PI polyamides can be produced conveniently by an automated synthesis method comprising solid-phase synthesis utilizing Fmoc (9-fluorenylmethoxycarbony) (Fmoc solid-phase synthesis method). The PI polyamides can be also produced by liquid-phase synthesis methods.

### 2. PI Polyamide Conjugates

As used herein, the term “conjugate” (complex) means two or more molecules forming a stable and bigger construct in which the molecules are linked via a bond (e.g. covalent bond) enough to form the stable and bigger construct. In an embodiment of the present disclosure, the PI polyamide is conjugated with a BD inhibitor at its N-terminus, C-terminus and/or γ-linker moiety to form a Bi-PI polyamide conjugate (Bi-PIP).

As used herein, the term “bromodomain (BD)” generally means a protein domain that selectively recognizes and binds to acetylated lysine. It is known that bromodomain is present in various proteins including HATs. For example, bromodomain recognizes acetylated lysine on histones.

As used herein, the term “BD inhibitor” means a compound that specifically interacts with bromodomain. Various compounds that specifically interact with bromodomain have been identified in recent years, and methods for such identification have been also developed. For example, in an embodiment of the present disclosure, the BD inhibitor is a compound that specifically binds to the pocket of BD to prevent binding with an acetylated lysine residue. As used herein, the BD inhibitor does not necessarily have to produce inhibitory effect, as long as it specifically binds to BD. The BD inhibitor may be a compound that produces interaction other than inhibitory effect.

In an aspect of the present disclosure, the BD inhibitor contained in the PI polyamide conjugate is not particularly limited, and it is preferably selected from compounds that specifically bind to BD of BD-containing proteins having desired biochemical activity. Such a BD inhibitor may be identified by a known method. Examples of the BD-containing proteins include, but not limited to, various enzymes and transcriptional activators, and specifically HATs, histone methyltransferases, ATP-dependent chromatin-remodeling complex proteins, and BET (bromodomain and extra-terminal) proteins (e.g., BRD2, BRD3, BRD4, BRDT etc.). Examples of HATs having both HAT domain and BD include P300/CBP family such as P300 (KAT₃B), CBP (KAT₃A), etc., GNAT family such as GCN5 (KAT₂A), PCAF (KAT₂B), etc., and TAF1 (KAT₄).

Specific examples of the BD inhibitor include, but not limited to, 5-isoxazolyl-benzimidazole compounds, JQ1 and their similar compounds, I-BET762, and OTX015. In an aspect of the present disclosure, a known BD inhibitor may be used, or a BD inhibitor identified by a known method may be used.

Examples of 5-isoxazolyl-benzimidazole compounds as the BD inhibitors include, but not limited to, compounds represented by the following formula:

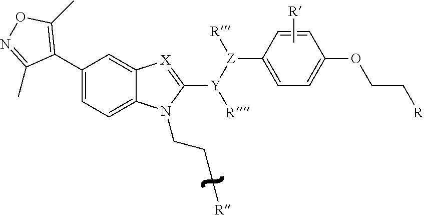

wherein,

R is NH₂ or NHCOCH₂CH₂CH₂COOH,

R′ is H, CH₃, F, Cl, or NO₂,

R″ is a group represented by the following formula:

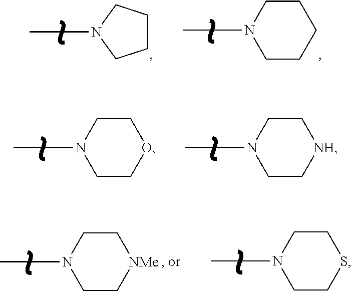

R′″ is H or CH₃,

R″″ is H or CH₃,

X is CH or N,

Y is CH or O, and

Z is CH or O.

Specific examples of 5-isoxazolyl-benzimidazole compounds as the BD inhibitors include, but not limited to, compounds represented by the following formula:

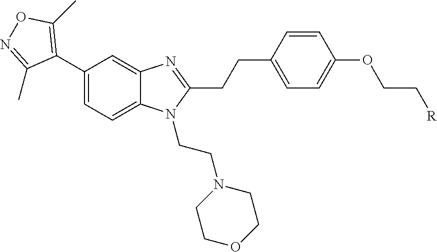

wherein R is NH₂ or NHCOCH₂CH₂CH₂COOH.

Conjugation (binding) between the BD inhibitor and the PI polyamide can be performed by a conventional method (see, e.g., J. Am. Chem. SOC. 1995, 117, 2479-2490). The BD inhibitor may be bound to the N terminus, C terminus, and/or γ linker moiety of the PI polyamide directly or via a linker. When the BD inhibitors are bound to two or more positions selected from the N terminus, C terminus and γ linker moiety, the BD inhibitors may be the same or different from each other.

The linker between the BD inhibitor and the PI polyamide in the PI polyamide conjugate is not particularly limited as long as the linker interferes with neither the action of the BD inhibitor nor the DNA sequence recognition by the PI polyamide. Examples of the linker include bonds themselves such as an amide bond, a phosphodisulfide bond, an ester bond, a coordinate bond, an ether bond and the like, and a molecule containing a functional group that forms at least one type of the bonds. The “molecule containing a functional group that forms at least one type of the bonds” is a molecule containing a functional group that forms at least one type of bonds selected from the group consisting of an amide bond, a phosphodisulfide bond, an ester bond, a coordinate bond, an ether bond and the like, along with the terminal portion of the PI polyamide and/or the BD inhibitor. The “molecule containing a functional group that forms at least one type of the bonds” may be a molecule containing one or more bonds being at least one type of bonds selected from the group consisting of an amide bond, a phosphodisulfide bond, an ester bond, a coordinate bond, an ether bond and the like. Preferred examples of the linker include an amide bond, and a molecule containing a functional group that forms an amide bond. The linker can be appropriately determined by a person skilled in the art.

In an aspect of the present disclosure, the PI polyamide conjugates may be in the form of a pharmacologically acceptable salt. Examples of the pharmacologically acceptable salt include, but not limited to, inorganic acid salts such as hydrochloride, sulfate, phosphate and hydrobromide, and organic acid salts such as acetate, fumarate, maleate, oxalate, citrate, methanesulfonate, benzenesulfonate and toluenesulfonate.

In the PI polyamide conjugate, at least one moiety or molecule of the PI polyamide, the BD inhibitor, and/or the linker moiety linking the PI polyamide and the BD inhibitor may be present in the form of an enantiomer or diastereomer or a mixture thereof. The PI polyamide conjugate includes a mixture of stereoisomers, or a pure or substantially pure isomer thereof. When the PI polyamide conjugate is obtained in the forms of diastereomers or enantiomers, these diastereomers or enantiomers can be separated by a conventional method well known in the art, e.g., chromatography or fractional crystallization.

The PI polyamide conjugate may be labeled with a radioisotope (e.g., ³H, ¹³C, ¹⁴C, ¹⁵N, ¹⁸F, ³²P, ³⁵S, ¹²⁵I, or the like) or the like on at least one moiety or molecule of the PI polyamide, the BD inhibitor, and/or the linker moiety linking the PI polyamide and the BD inhibitor, or may be deuterated.

### 3. Recruitment of Bromodomain-Containing Proteins

Since the PI polyamide conjugate includes a PI polyamide that recognizes a specific DNA sequence and a BD inhibitor that targets a specific BD-containing protein as described above, the PI polyamide conjugate binds to the specific sequence on a DNA through the DNA sequence recognition by the PI polyamide and at the same time, recruits the target BD-containing protein through the BD inhibitor. Thus, a BD-containing protein having a desired biochemical activity is recruited to a desired specific region on genomic DNA or a specific nucleosome containing the desired region by utilizing the PI polyamide conjugate, and consequently, the desired biochemical activity of the BD-containing protein is led to the specific region or nucleosome. Examples of the biochemical activity include, but not limited to, posttranslational modifications, e.g., modifications, such as acetylation or methylation, of histones. Thus, the present disclosure provides a composition for recruiting a BD-containing protein to a specific region on DNA or a specific nucleosome, i.e. a composition for DNA sequence-selectively recruiting a BD-containing protein (hereinafter also referred to as “the BD-containing protein-recruiting composition”). Further, the present disclosure provides a method of recruiting a BD-containing protein to a specific region on DNA or a specific nucleosome, i.e. a method of DNA sequence-selectively recruiting a BD-containing protein (hereinafter also referred to as “the BD-containing protein-recruiting method”). Further, the present disclosure provides a composition for regulating biochemical activity in a specific region on DNA or a specific nucleosome (hereinafter also referred to as “the biochemical activity-regulating composition”). Further, the present disclosure provides a composition for regulating histone modification in a specific region on DNA or a specific nucleosome (hereinafter also referred to as “the histone modification-regulating composition”). As used herein, the term “regulation”, “regulating” or “regulate” includes “activation” and “suppression”, “activating” and “suppressing” or “activate” and “suppress”, respectively. Compounds that provide desired regulation can be produced depending on the types of BD-containing proteins targeted by the PI polyamide conjugates.

The bromodomain-containing protein-recruiting composition, the biochemical activity-regulating composition and the histone modification-regulating composition (hereinafter also referred to as “the BD-containing protein-recruiting composition and the like” collectively) are compositions containing the PI polyamide conjugates disclosed herein. The BD-containing protein-recruiting composition and the like may be the PI polyamide conjugates themselves; or may contain not only the PI polyamide conjugates but also carriers, additives or both of them depending on the intended uses. The carriers and additives are preferably pharmacologically acceptable carriers and additives. Examples of the carriers and the additives include, but not limited to, water, acetic acid, organic solvents, collagen, polyvinyl alcohol, polyvinylpyrrolidone, carboxyvinyl polymers, sodium carboxymethylcellulose, sodium polyacrylate, sodium alginate, water-soluble dextran, sodium carboxymethyl starch, pectin, methylcellulose, ethylcellulose, xanthan gum, gum arabic, casein, agar, polyethylene glycol, diglycerol, glycerol, propylene glycol, Vaseline, paraffin, stearyl alcohol, stearic acid, human serum albumin, mannitol, sorbitol, lactose, and surfactants. The BD-containing protein-recruiting composition and the like can be formulated into desired dosage forms according to conventional methods depending on the intended use. Such dosage forms include dosage forms for oral administration and dosage forms for parenteral administration. Examples of the dosage forms include, but not limited to, tablets, capsules, fine granules, powders, granules, solutions, syrups, sprays, liniments, eye drops, preparations for external use, and injections.

The BD-containing protein-recruiting method comprises bringing the PI polyamide conjugate or the bromodomain-containing protein-recruiting composition disclosed herein into contact with a sample. The method may be performed in vitro, in vivo, or ex vivo. The sample contains a DNA, e.g., a genomic DNA, or a nucleosome, and includes a living body and a biological sample. Examples of the living body include all organisms that utilize double-stranded DNAs in biocontrol, such as mammals (e.g., human, rat, rabbit, sheep, pig, cattle, cat, dog, monkey, etc.). The biological sample means any sample isolated from a living body. Examples of the biological sample include, but not limited to, blood, body fluid, and various cells. When a cell is used as the sample, e.g. the PI polyamide conjugate or the bromodomain-containing protein-recruiting composition disclosed herein is added to a culture medium and the cell is cultured in the medium. The addition amount of the PI polyamide conjugate or the bromodomain-containing protein-recruiting composition and the culture conditions can be appropriately determined by a person skilled in the art.

### 4. Induction and Promotion of Histone Acetylation

When the PI polyamide conjugate contains an inhibitor against BD of HAT, the PI polyamide conjugate can induce and promote histone acetylation in a specific nucleosome. In other words, the PI polyamide conjugate binds to a specific sequence on a DNA through the DNA sequence recognition by the PI polyamide, while the PI polyamide conjugate leads the target HAT of the BD inhibitor to a nucleosome containing the specific sequence through the BD inhibitor and then acetylates a lysine residue on a histone near the specific sequence. Once the histone acetylation is induced by the PI polyamide conjugate, BD contained in HAT binds to the acetylated lysine and lysine residues near the acetylated lysine are then acetylated. Such HAT-mediated propagation of acetylation promotes histone acetylation. Thus, the PI polyamide conjugate comprising a BD inhibitor against HAT promotes acetylation of nucleosomes by utilizing the BD-mediated natural propagation system of histone acetylation for the control of targeted acetylation. Therefore, according to the present disclosure, a composition for inducing and promoting histone acetylation of nucleosomes in a DNA sequence-selective manner, and a method of acetylating nucleosomes in a DNA sequence-selective manner are provided.

In an aspect of the present disclosure, a composition for inducing histone acetylation, which comprises the PI polyamide conjugate disclosed herein (hereinafter, also referred to as “the histone acetylation-inducing composition”) is provided. Further, the histone acetylation-inducing composition promotes histone acetylation. The histone acetylation-inducing composition may be the PI polyamide conjugate itself, or may contain not only the PI polyamide conjugate but also carriers, additives or both of them depending on the intended use. Examples of the carriers and additives are the same as cited above for the BD-containing protein-recruiting composition and the like. The histone acetylation-inducing composition can be formulated into desired dosage forms according to conventional methods depending on the intended use. Examples of the dosage forms are the same as cited above for the BD-containing protein-recruiting composition and the like.

In an aspect of the present disclosure, a method of acetylating nucleosomes which comprises bringing the PI polyamide conjugate or the histone acetylation-inducing composition disclosed herein into contact with a sample containing the nucleosomes (hereinafter also referred to as “the nucleosome acetylation method”) is provided. The nucleosome acetylation method may be performed in vitro, in vivo, or ex vivo. Examples of the sample are the same as cited above for the BD-containing protein-recruiting method. Since the PI polyamide conjugate includes the PI polyamide that recognizes a specific sequence, the nucleosome acetylation method acetylates histones in nucleosomes in a sequence-selective manner.

In fact, when a mixture of nucleosomes was reacted with HAT (P300) in the presence of Bi-PIP, a nucleosome containing a target DNA sequence was selectively acetylated, indicating that sequence-selective acetylation was promoted (see Example 2). Further, cultured cells (HEK293T cells) were used to demonstrate that DNA sequence-specific acetylation selectively upregulates gene expression (see Example 4). In the present disclosure, any desired gene group can be activated by varying the recognition sequence of the PI polyamide.

Hereinafter, the novel techniques disclosed herein are further specifically explained by way of Examples which should not be interpreted to limit the scope of the said novel techniques.

### EXAMPLES

### Example 1: Design and Synthesis of Bi-PIP

### (1) Design and Synthesis of BD Inhibitors

As the targeted BD-containing protein, coactivator P300/CBP family of proteins was selected. P300/CBP is HAT that plays a central role in transcriptional activation in eukaryotic cells. P300/CBP has both HAT domain and BD. A P300/CBP-selective BD inhibitor (CBP30) having 5-isoxazolyl-benzimidazole was previously reported (Hay et al., J. Am. Chem. Soc. 2014, 136, 9308-9319). As the BD inhibitor, one of the CBP30 derivatives which showed a moderate binding affinity to the BD of CBP was used. The BD inhibitor was synthesized according to the previous report (Hay et al., supra). Then, a BD inhibitor unit with primary amine was designed and synthesized by a conventional method.

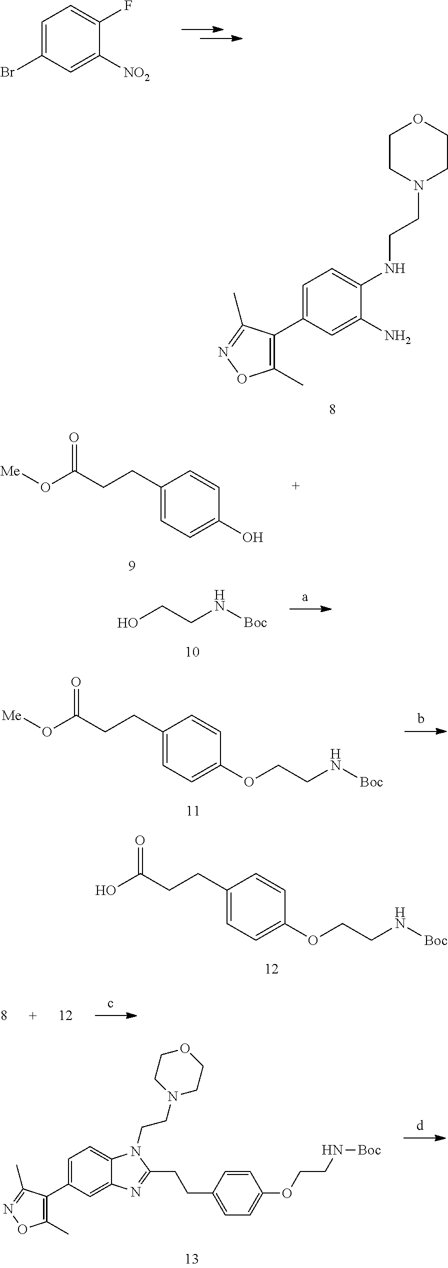

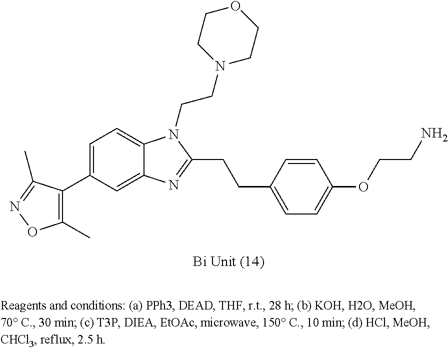 [SMILES: Cc1noc(C)c1-c1ccc2c(c1)nc(CCc1ccc(OCCN)cc1)n2CCN1CCOCC1]

### Methyl 3-(4-(2-((tert-butoxycarbonyl)amino)ethoxy)phenyl)propanoate (11)

Methyl 3-(4-hydroxyphenyl)propanoate (9) and tert-butyl (2-hydroxyethyl)carbamate (10) were synthesized by a known method. Methyl 3-(4-hydroxyphenyl)propanoate (550 mg, 3.1 mmol), tert-butyl (2-hydroxyethyl)carbamate (486 mg, 3.0 mmol) and triphenylphosphine (PPh₃) (1.01 g, 3.9 mmol) were dissolved in THF (3 mL) and cooled to 4° C. Diethyl azodicarboxylate (DEAD) (690 mg, 4.0 mmol) in THF (3 mL) was drop-wisely added. The reaction mixture was then warmed to room temperature and stirred overnight. The mixture was then concentrated and dissolved in ethyl acetate (EtOAc). The organic phase was washed with water, aqueous sodium hydroxide (NaOHaq) and brine. The solution was dried with Na₂SO₄, filtered, and concentrated. The crude compound was purified by a silica-gel column chromatography (hexane/EtOAc=4:1) to give 510 mg of colorless oil (1.6 mmol, 53%).

¹H NMR (600 MHz, CDCl₃): δ=7.11 (d, J=8.3 Hz, 2H), 6.81 (d, J=8.9, 2H), 4.98 (s, br, 1H), 3.99 (t, J=4.8 Hz, 2H), 3.66 (s, 3H), 3.52 (d, J=5.5 Hz, 2H), 2.89 (t, J=7.9 Hz, 2H), 2.59 (t, J=7.9, 2H), 1.45 (s, 9H).

### Methyl 3-(4-(2-((tert-butoxycarbonyl)amino)ethoxy)phenyl)propionic acid (12)

Compound 11 (510 mg, 1.6 mmol) and potassium hydroxide (525 mg, 9.36 mmol) were dissolved in a mixture of MeOH (10 mL) and water (5 mL). The mixture was stirred at 70° C. for minutes and then cooled to room temperature. The mixture was neutralized with AcOH, extracted twice with EtOAc and washed with brine. The mixture was then dried with Na₂SO₄, filtered and concentrated to give 136 mg of white solid (0.44 mmol, 27%).

¹H NMR (600 MHz, CDCl₃): δ=7.12 (d, J=8.9 Hz, 2H), 6.82 (d, J=8.2 Hz, 2H), 4.99 (s, br, 1H), 3.99 (t, J=5.1 Hz, 2H), 3.52 (m, 2H), 2.90 (t, J=7.6 Hz, 2H), 2.65 (t, J=7.6 Hz, 2H), 1.45 (s, 9H).

### tert-butyl (2-(4-(2-(5-(3,5-dimethylisoxazol-4-yl)-1-(2-morpholinoethyl)-1H-benzo[d]imidazol-2-yl)ethyl)phenoxy)ethyl)carbamate (13)

3-(4-(2-((tert-butoxycarbonyl)amino)ethoxy)phenyl)propanoic acid (8) was synthesized by a known method. Compound 8 (94 mg, 0.30 mmol), compound 12 (93 mg, 0.30 mmol), propylphosphonic acid anhydride (T3P) (193 mg, 0.61 mmol) and N,N-diisopropylethylamine (DIEA) (63 μL, 0.36 mmol) were dissolved in EtOAc (4 mL). The mixture was heated in a microwave (150° C., 10 minutes). The reaction was basified with 0.1 M NaOHaq and partitioned between EtOAc and water. The organic phase was collected and washed with brine. The mixture was dried with MgSO₄ and filtered. The crude compound was purified by a silica-gel column chromatography (CH₂Cl₂/MeOH=9:1) to give 132 mg of beige gum (0.22 mmol, 73%).

¹H NMR (600 MHz, CDCl3): δ=7.63 (s, 1H), 7.35 (d, J=8.3, 1H), 7.15 (d, J=8.9, 2H), 7.12 (d, J=7.9, 1H), 6.84 (d, J=8.9, 2H), 4.98 (s, br, 1H), 4.12 (m, 2H), 4.00 (t, J=5.2, 2H), 3.67 (t, J=4.5, 4H), 3.53 (d, J=4.8, 2H), 3.23 (m, 2H), 3.17 (m, 2H), 2.61 (t, J=7.2, 2H), 2.46 (t, 4H), 2.43 (s, 3H), 2.30 (s, 3H), 1.45 (s, 9H)

### 2-(4-(2-(5-(3,5-dimethylisoxazol-4-yl)-1-(2-morpholinoethyl)-1H-benzo[d]imidazol-2-yl)ethyl)phenoxy)ethan-1-amine (Bi Unit, 14)

Compound 13 (30 mg, 51 μmol) was dissolved in a mixture of MeOH (5 mL) and CH₂Cl₂ (5 mL) containing 1.25 mmol of HCl. The mixture was refluxed for 3 hours. The reaction was cooled, concentrated, and dissolved in EtOAc and CH₂Cl₂. The mixture was neutralized with saturated NaHCO₃ aq. The organic phase was washed with brine, dried with Na₂SO₄, and filtered. The mixture was then concentrated to give 25.6 mg of beige gum. The compound was used for the next reaction without further purification.

### (2) Design and Synthesis of PI Polyamides

Two PI polyamides targeting 5′-WWCWGWCW-3′ and 5′-WWCWGWCW-3′ (W=A or T) (referred to as “PIP1” and “PIP2” respectively) were designed and synthesized by a known method. Briefly, solid phase synthesis was performed on Py-oxime resin by PSSM-8 peptide synthesizer (Shimadzu, Japan). The compound was then cleaved with N,N-dimethylpropanediamine (45° C., 3 h). The crude compound was purified with reversed phase HPLC on an Engineering PU-2089 plus series system (Jasco) utilizing a YMC Pack Pro C18 column (150×20 mm, YMC). 0.1% TFA in water and acetonitrile were used as the eluent at a flow rate of 10 ml/min with detection at 254 nm.

### (3) Synthesis of PI Polyamide Conjugates

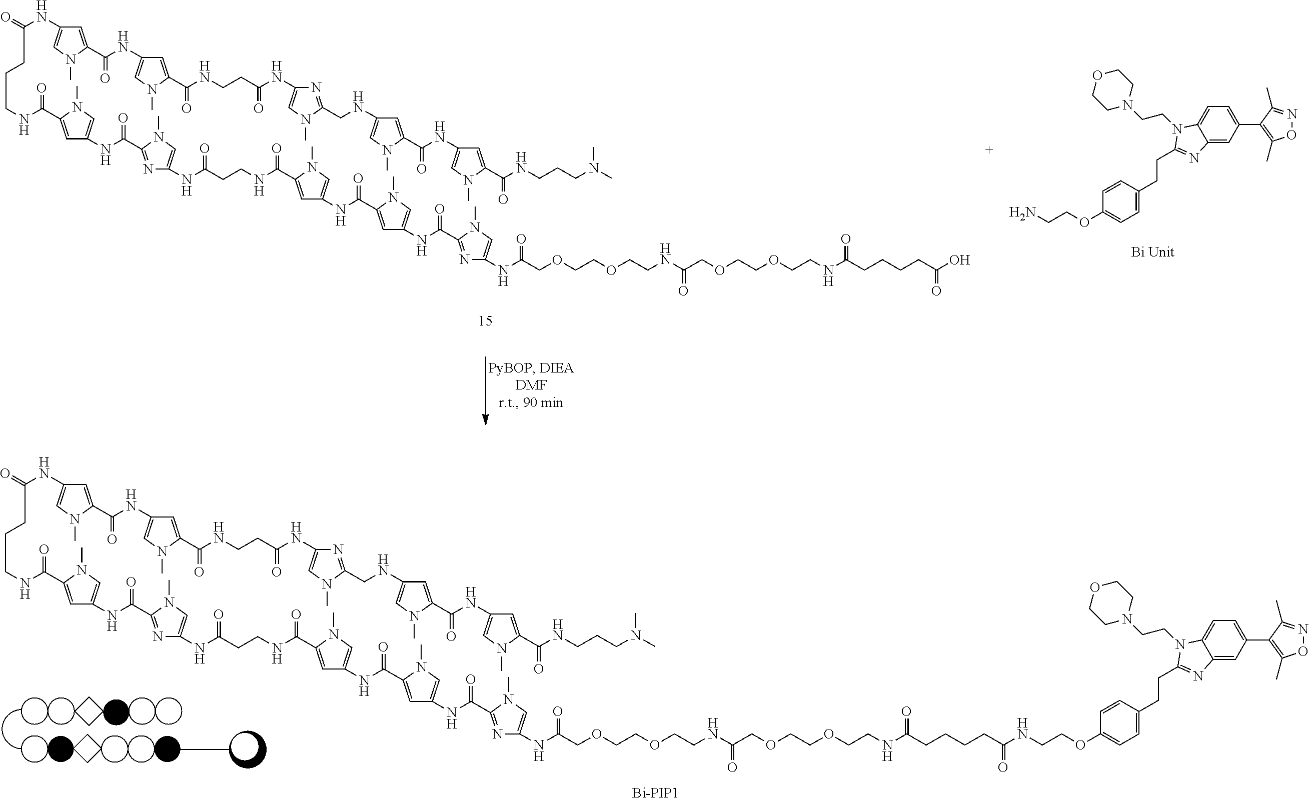

By coupling of the BD inhibitor unit with the PIP as obtained above, which had a carboxylic acid at the N-terminus, two conjugates targeting 5′-WWCWGWCW-3′ and 5′-WWCWGWCW-3′ (referred to as “Bi-PIP1” and “Bi-PIP2” respectively) were synthesized by a known method. Briefly, Bi Unit (1 eq.) and PIP (1 eq.) and 1H-Benzotriazol-1-yloxy-tri(pyrrolidino)phosphonium hexafluorophosphate (PyBOP) (2 eq.) were mixed in 150 μL of dimethylformamide. DIEA (5 eq.) was then added and the reaction was shaken at room temperature for 1.5 hour. The crude Bi-PIPs were purified with reversed phase HPLC on an Engineering PU-2089 plus series system (Jasco) utilizing a COSMOSIL 5C18-MS-II column (150×10 mm, Nacalai Tesque). 0.1% TFA in water and acetonitrile were used as the eluent at a flow rate of 3 ml/min with detection at 254 nm. Purified Bi-PIPs were characterized by analytical HPLC and MALDI-TOF MS.

Bi-PIP1

Analytical HPLC_{tR}=17.6 min; MALDI-TOF MS m/z calcd for C₁₁₈H₁₅₂N₃₅O₂₄⁺ [M+H]⁺ 2443.17, found 2443.51.

Bi-PIP2

Analytical HPLC_{tR}=17.8 min; MALDI-TOF MS m/z calcd for C₁₁₅H₁₄₉N₃₈O₂₄⁺ [M+H]⁺ 2446.16, found 2446.46

### Example 2: Promotion of Histone Acetylation on Nucleosomes Having Target DNA Sequences by Bi-PIP

### (1) Sequence-Selective Histone Acetylation on Nucleosomes by Bi-PIP

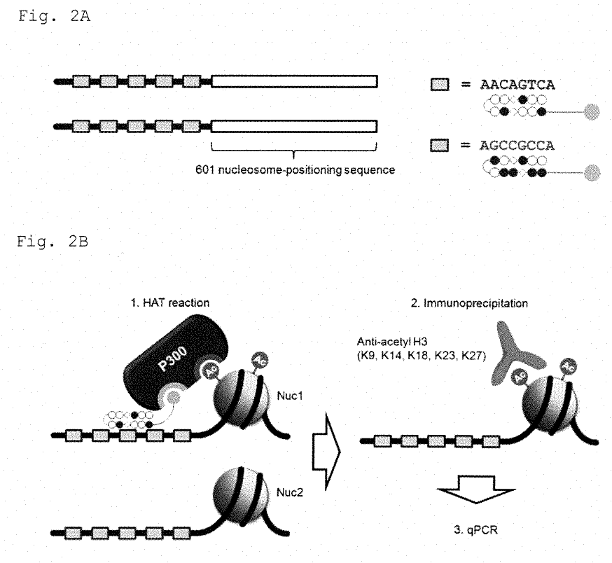

To test sequence-selective histone acetylation by Bi-PIP, a biochemical assay (HAT reaction-in vitro ChIP-qPCR) was established by combining HAT reaction followed by immunoprecipitation utilizing reconstituted nucleosomes as reported previously (Nguyen et al., Nat. Methods 2014, 11, 834-40) and quantitative polymerase chain reaction (qPCR) (FIGS. 2A to 2C).

Histone Expression and Purification

Human histone proteins (H2A type1-B/E, H2B type1-K, H3.1 and H4) were expressed in Escherichia coli BL21 (DE3) utilizing a T7 promoter-driven expression vector, pET22b. Crude histones in a urea buffer (6 M urea, 20 mM HEPES-KOH (pH 7.5) and 1 mM 2-mercaptoethanol) were purified by positive ion exchange chromatography utilizing a HiTrap SP HP column (GE Healthcare) on AKTA pure 25 protein purification system (GE Healthcare). A urea buffer containing 0.2-0.7 M NaCl was used for the elution. Fractions containing target histone monomer were collected and concentrated utilizing Amicon Ultra-4 (Merck), MWCO 10 kDa centrifugal filter unit. The buffer was exchanged with an unfolding buffer (6 M guanidium chloride, 20 mM HEPES-KOH (pH 7.5) and 5 mM 2-mercaptoethanol).

Preparation of Histone Octamer

10 ml of the H2A-H2B-H3-H4 octamer reconstitution solution containing 20 μM of each histone monomer in the unfolding buffer as obtained above was packed in 7000 MWCO SnakeSkin Dialysis Tubing (Thermo Fisher Scientific) and dialyzed against 500 ml of a pre-refolding buffer (6 M urea, 2 M NaCl, 10 mM HEPES-KOH (pH 7.5), 1 mM EDTA and 5 mM 2-mercaptoethanol) and a refolding buffer (pre-refolding buffer without 6 M urea) for 2 hours and 12 hours respectively at 4° C. After impurities were removed by centrifugation (15000 rpm, 10 min, 4° C.), the histone octamer was purified by size exclusion chromatography utilizing Superdex 200 Increase 200 (GE Healthcare) and the refolding buffer on AKTA pure 25 protein purification system. Fractions were analyzed by SDS-PAGE and those containing histone octamer were collected and concentrated utilizing Amicon Ultra-4.

Nucleosome Reconstitution

Mononucleosomes were reconstituted utilizing a salt-dialysis method from a histone octamer and DNA templates that each have a Widom 601 nucleosome positioning sequence and five PIP-binding sequences: 5′-AACAGTCA-3′ for Bi-PIP1 and 5′-AGCCGCCA-3′ for Bi-PIP2 (FIG. 2A). Gel electrophoresis confirmed that the majority of DNA templates were successfully formed on the nucleosome. Two types of nucleosomes thus obtained, one with five match sequences for Bi-PIP1 and the other with five match sequences for Bi-PIP2, were referred to as “Nuc1” and “Nuc2” respectively.

Briefly, a mixture of template DNA (400 nM) and histone octamer (440 nM) in a 20 mM HEPES-KOH buffer (pH 7.5) was dialyzed utilizing a dialysis cup (MWCO 8000, Bio-tech) in high-salt buffer (20 mM HEPES-KOH, pH 7.5; 2 M NaCl) for 2 hours, then in a low-salt buffer (20 mM HEPES-KOH, pH 7.5; 0 M NaCl) overnight at 4° C. After the dialysis, the concentration of nucleosome was determined by measuring the absorbance of DNA at the wave length of 260 nm. The reconstituted nucleosomes were analyzed by polyacrylamide gel electrophoresis (6% DNA retardation gel, Thermo Fisher Scientific). The sequences of the DNA templates used in this Example were shown below.

Template DNA for Nuc1:

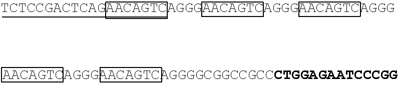

TGCCGAGGCCGCTCAATTGGTCGTAGACAGCTCTAGCACCGCTTAA
ACGCACGTACGCGCTGTCCCCCGCGTTTTAACCGCCAAGGGGATTA
CTCCCTAGTCTCCAGGCACGTGTCAGATATATACATCCTGT

A forward primer and a reverse primer are underlined. Bi-PIP binding sequences are boxed off. Bold letters indicate the Widom 601 nucleosome positioning sequence.

Template DNA for Nuc2:

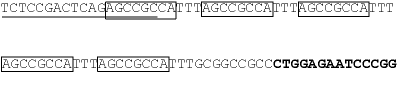

TGCCGAGGCCGCTCAATTGGTCGTAGACAGCTCTAGCACCGCTTAA
ACGCACGTACGCGCTGTCCCCCGCGTTTTAACCGCCAAGGGGATTA
CTCCCTAGTCTCCAGGCACGTGTCAGATATATACATCCTGT

A forward primer and a reverse primer are underlined. Bi-PIP binding sequences are boxed off. Bold letters indicate the Widom 601 nucleosome positioning sequence.

Histone Acetylation Reaction

HAT reaction was performed according to a known method. Briefly, a mixture of reconstituted nucleosomes Nuc1 and Nuc2 (10 nM each) in 15 μL of a HAT buffer (50 mM Tris-HCl, 0.1 mM EDTA, 10% glycerol, 1 mM DTT, pH 8.0) was incubated with recombinant human P300 (BPS Bioscience, catalog number: 50071) at various concentrations (0, 1, 3, 10, 30, 100 nM) in the presence of 10 μM of acetyl-CoA (Wako), with or without 100 nM Bi-PIPJ for 1 hour at 30° C. When 100 nM Bi-PIP1 was not added, DMSO (vehicle) was added instead.

In Vitro Chromatin Immunoprecipitation to Quantitative Polymerase Chain Reaction (In Vitro ChIP-qPCR)

To a HAT reaction (15 μL), 85 μL of an AB buffer (20 mM Tris-HCl, pH 7.5, 50 mM NaCl, 5 mM EDTA) containing 1/500 volume of anti-acetylated H3 (Abcam, ab47915) was added and rotated for 1 hour at room temperature. Then, 40 μL of Dynabeads Protein G (Thermo Fisher Scientific) in 100 μL of the AB buffer was added to the mixture and further rotated for 1 hour at room temperature. The beads were washed three times with 200 μL of the AB buffer and 100 μL of an Elution buffer (100 mM Tris-HCl, pH 7.5, 1 M NaCl) was added. The precipitated DNA was eluted by shaking at 90° C. for 5 minutes. The DNA solution was diluted, and amount of the precipitated each nucleosome was measured by qPCR utilizing SYBR FAST qPCR Kit (Kapa Bioscience) on LightCycler 480 (Roche Diagnostics GmbH) (FIG. 2B). Cp values were determined by the 2nd derivative maximum method and relative RNA amount was calculated by the ΔΔCp method. For qPCR, primers specific for the DNA templates were used (Table 1). As positive control, the same experiment was performed utilizing a nucleosome with synthetic acetylated histone H3 (K4ac, K9ac, K14ac, K18ac, K23ac).

TABLE 1

| | Forward/ | |
|---|---|---|
| Target | Reverse | Sequence (5′-3′) |
| Nuc1 | F | TCTCCGACTCAGAACAGTC |
| Nuc2 | F | TCTCCGACTCAGAGCCGC |
| Nuc1 & Nuc2 | R | GCACCGGGATTCTCCAG |
| (common) | | |

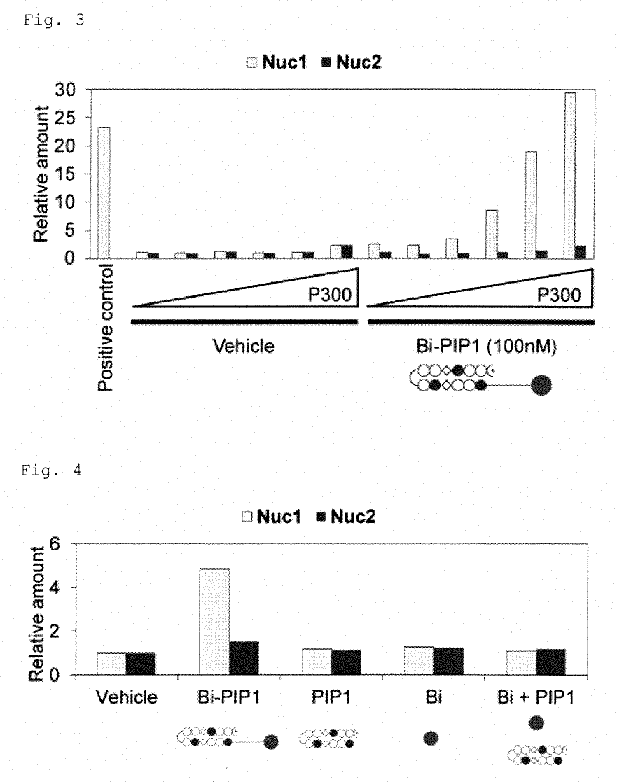

Results are shown in FIG. 3. Addition of 100 nM Bi-PIP1 induced intensive acetylation on its target Nuc1. On the other hand, acetylation was not induced on Nuc2. The increase in the level of acetylation was dependent on the concentration of P300.

(2) Comparison with Bi Monomer and PIP Monomer

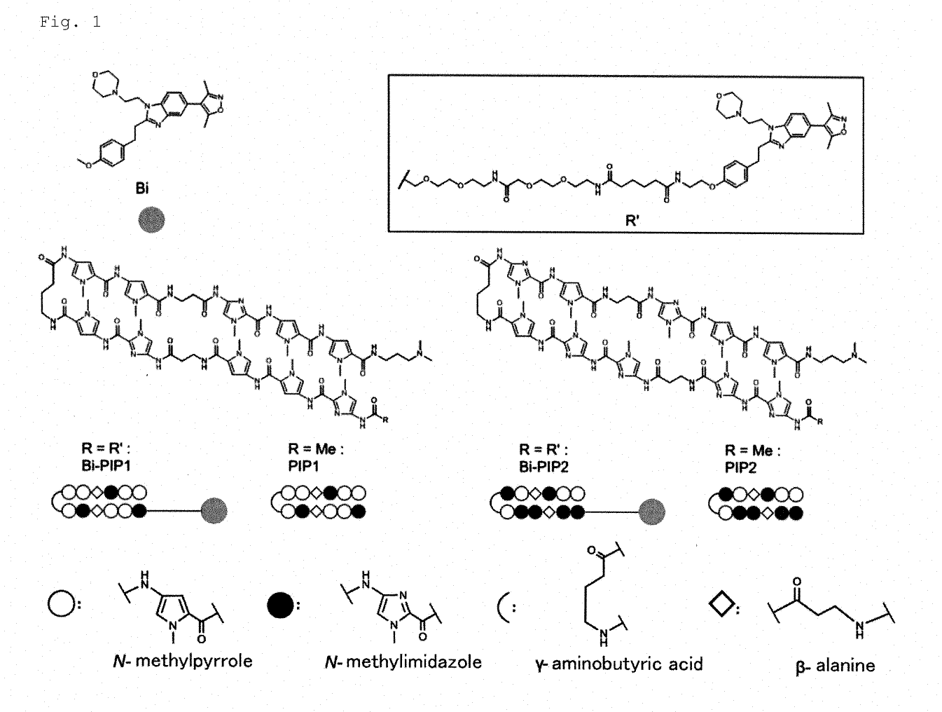

Next, to clarify the importance of the Bi and PIP domains, each monomer molecule was tested in the assay as described in above (1) (see FIG. 1). A mixture of Nuc1 and Nuc2 was incubated with 10 nM recombinant human P300 (amino acids 965-1810) in the presence of 100 nM Bi-PIP1, 100 nM PIP1 monomer alone, 100 nM Bi monomer alone, or a mixture of 100 nM PIP monomer and 100 nM Bi monomer, and subjected to ChIP and then qPCR, according to the method as described in above (1). As negative control, the same experiment was performed except that DMSO (vehicle) was added instead of the BI-PIP, PIP monomer or Bi monomer or the mixture of PIP monomer and Bi monomer.

Results are shown in FIG. 4. The PIP monomer alone, Bi monomer alone, and even a mixture of them did not promote the acetylation. These results clearly indicate that the Nuc1-selective acetylation by Bi-PIP1 was achieved by sequence-selective binding of the PIP domain to its target DNA sequence and P300 recruitment mediated by a BD-Bi interaction.

### (3) Confirmation of Programmability of Bi-PIP

To examine the programmability of Bi-PIP, the same experiment as described in above (1) was performed using Bi-PIP2 designed to target Nuc2 (see FIG. 1). A mixture of Nuc1 and Nuc2 was incubated with 10 nM recombinant human P300 (amino acids 965-1810) in the presence of 100 nM Bi-PIP1 or 100 nM Bi-PIP2, and subjected to ChIP and then qPCR, according to the method as described in above (1).

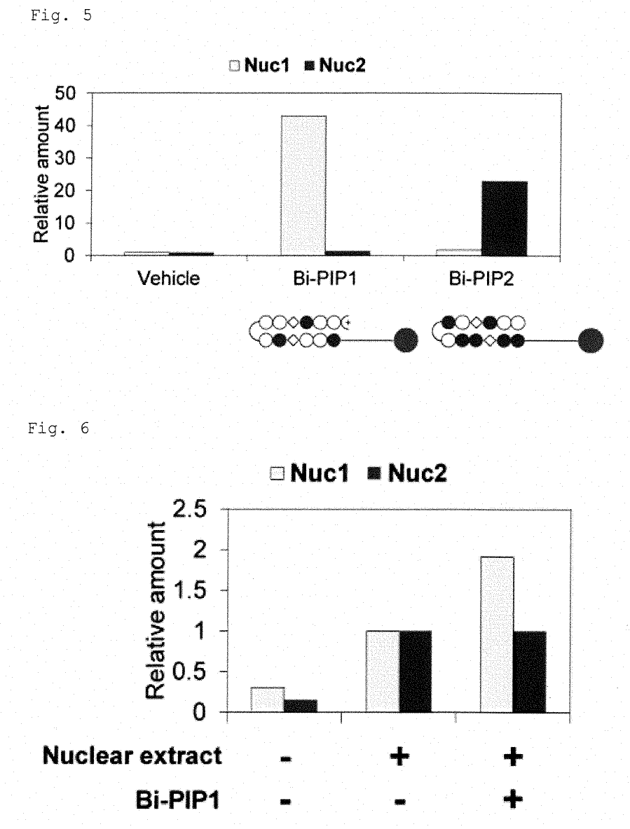

Results are shown in FIG. 5. Bi-PIP2 caused selective acetylation of Nuc2, indicating that the target sequence of Bi-PIP is programmable.

### (4) Mass Spectrometry Analysis

Histone acetylation with Bi-PIP was directly confirmed by mass spectrometry analysis. After HAT reaction using Nuc1 (500 nM), B PIP1 (0 or 2.5 μM), recombinant human P300 (50 nM) and Ac-CoA (10 μM), the histones were processed through propionylation of unmodified lysine, tryptic digestion, and propionylation of terminal amine. The resulting peptides were analyzed by liquid chromatography-tandem mass spectrometry (LC-MS/MS). Consistent with the antibody-based assays shown above, an increase in the signal intensity was observed for peptides containing acetyl lysine at the N-terminal tail region of H3, such as H3K14ac, H3K18ac, H3K23ac, H3K27ac, and H3K36ac. It was also found that acetylation was promoted in the N-terminal region of histone H4, including H4K5ac, H4K8ac, and H4K12ac.

### Example 3: Selective Gene Expression Activation Inside Living Cells by Bi-PIP

To evaluate if Bi-PIPs could function in a cellular environment, experiments were performed. As an initial experiment, a HAT reaction-in vitro ChIP-qPCR assay was performed using a HeLa nuclear extract as the HAT resource instead of recombinant P300 according to the method as described in Example 2-(1). The HeLa nuclear extract containing not only P300/CBP but also other cellular HATs and HDACs provided a cell-like environment similar to that observed with only recombinant P300. Briefly, the HeLa nuclear extract (0.3 μL/reaction) was added to a mixture of Nuc1 and Nuc2 up to 2% v/v in the reaction mixture. The reaction mixture was incubated with or without 100 nM Bi-PIP1, and subjected to ChIP and then qPCR. As control, the same experiment was performed using neither the Hela nuclear extract nor Bi-PIP1.

Results are shown in FIG. 6. In FIG. 6, relative amounts of Nuc1 and Nuc2 normalized to the value of the [Nucelar extract (+)/Bi-PIPJ (−)] sample are shown. Although the basal level of acetylation without Bi-PIP1 increased, Bi-PIP1 enhanced acetylation of Nuc1 but not Nuc2, suggesting the possibility of applying Bi-PIPs to living cells.

### Example 4: Transcriptome Analysis in Cells Treated with Bi-PIPs

Considering the fundamental role of P300/CBP as the transcriptional coactivator to activate gene expression, transcriptome analyses of total RNA extracted from cells (HEK293T cells) treated with Bi-PIPs were performed. The criteria of >2-fold or <−2-fold change and <0.05P value was applied for detecting differentially expressed transcripts.

Cell Treatment and RNA Extraction

HEK293T line was purchased from ATCC and maintained in DMEM (Thermo Fisher Scientific) supplemented with 10% (v/v) fetal bovine serum (FBS, Sigma Aldrich) at 37° C. with 5% CO₂. For the Bi-PIP treatment, the cells were seeded on 24 well plates (2×10⁵ cells/well). After one day, 1 μM of each Bi-PIP and 4 μM of Endo-Porter (10% polyethylene glycol form, Gene Tools) were added. As a vehicle, 0.1% DMSO (v/v) was used. After 15 hours of Bi-PIP treatment, RNA was extracted using FastGene™ RNA Basic Kit (Nippon Genetics) following manufacture's instruction.

Microarray

The quality of RNA was checked with Bioanalyzer (Agilent Technologies) using Agilent RNA 6000 Pico Kit (Agilent Technologies). The total RNA was processed by GeneChip WT PLUS Reagent Kit (Affymetrix) and hybridized to Human Gene 2.1 ST Array Strip (Affymetrix) using GeneChip Hybridization, Wash, and Stain Kit (Affymetrix). Fluidics and scanning were conducted in GeneAtlas System (Affymetrix) using GeneChip Hybridization, Wash, and Stain Kit (Affymetrix). Data normalization, summarization and comparison were performed using Transcriptome Analysis Console v4 (Affymetrix).

As a result, the Bi monomer showed only a minimal change in gene expression (two upregulated transcripts and four downregulated transcripts). The PIP1 and PIP2 monomers caused a moderate change in global gene expression (32 upregulated and 31 downregulated by PIP1, and 143 upregulated and 63 downregulated by PIP2). On the other hand, Bi-PIP1 and Bi-PIP2 conjugates gave greater transcriptome changes, mainly for activation, rather than repression (473 upregulated and 258 downregulated by Bi-PIP1, and 446 upregulated and 137 downregulated by Bi-PIP2). Therefore, it was shown that Bi-PIPs recruited the P300/CBP onto the chromatin and histone acetylation caused activation of gene expression.

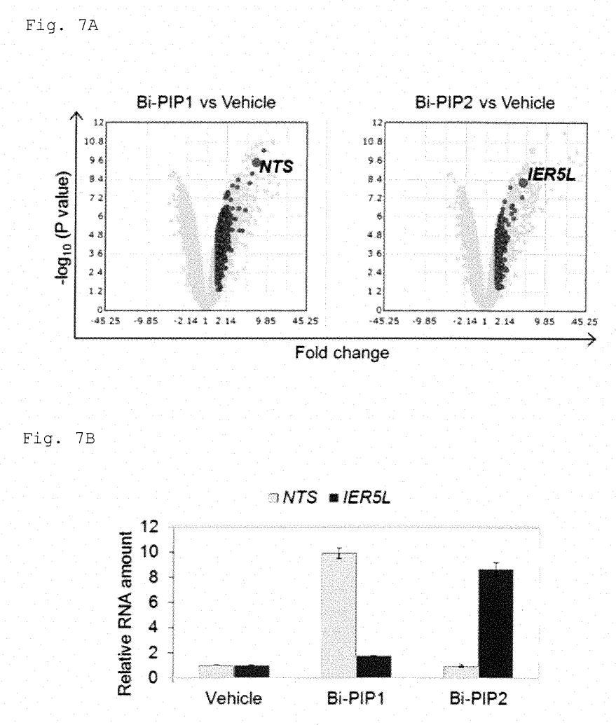

Then, whether the difference in DNA sequence-selectivity could cause a dissimilar change in gene expression was evaluated. As a result, 247 transcripts were commonly upregulated by both Bi-PIP1 and Bi-PIP2. This is reasonable because the binding sites of Bi-PIP1 and Bi-PIP2 are not exclusively distributed throughout the genome; thus, some genes could be activated by both Bi-PIP1 and Bi-PIP2. Although the results described above showed that some genes were activated by both Bi-PIP1 and Bi-PIP2, differentially activated transcripts were also identified in the microarray data by applying criteria of >1.5- or <−1.5-fold change (FIG. 7A). Among these uniquely activated transcripts, the most upregulated protein-coding genes with Bi-PIP1 and Bi-PIP2 were NTS (neurotensin) and IER5L (immediate early response 5-like), respectively.

RT-qPCR

The expression levels of the two transcripts were measured by using RT-qPCR. The total RNA was reverse transcribed by ReverTra Ace qPCR RT Master Mix with gDNA Remover (Toyobo). QPCR was performed using THUNDERBIRD SYBR qPCR Mix (Toyobo) in LightCycler 480 (Roche Diagnostics GmbH). Cp values were determined by the 2nd derivative maximum method and relative RNA amount was calculated by the ΔΔCp method. The primer sequences are listed in Table 2.

TABLE 2

| Gene | RefSeq | Forward/ | |
|---|---|---|---|
| Symbol | Accession | Reverse | Sequence (5′-3′) |
| GAPDH | NM_ | F | ACCACAGTCCATGCCATCAC |
| | 002046.6 | R | TCCACCACCCTGTTGCTGTA |
| NTS | NM_ | F | TGACCAATATGCATCATCAAAGA |
| | 006183.4 | R | TCTTGCAACAAGCTCCTCTTC |
| IER5L | NM_ | F | AAGATCCACAGCTCCCGAAC |
| | 203434.2 | R | GTAGCGCTCGCTCAGGTAG |

Results are shown in FIG. 7B. Further, the target sequences of Bi-PIP1 and Bi-PIP2 in each gene locus were examined. Interestingly, the genomic sequence of the NTS locus contains the binding sites for Bi-PIP1 but not for Bi-PIP2. Likewise, the IER5L locus possesses the binding sites for Bi-PIP2 but not for Bi-PIP1. From the distinct transcriptional activation and discrete binding site distribution described above, it was concluded that NTS and IER5L are putative target genes of Bi-PIP1 and Bi-PIP2, respectively.

### SEQUENCE FREE TEXT

SEQ ID NO:1: Sequence of template DNA for Nuc1
SEQ ID NO:2: Sequence of template DNA for Nuc2
SEQ ID NO:3: Forward primer for Nuc1
SEQ ID NO:4: Forward primer for Nuc2
SEQ ID NO:5: Reverse primer for Nuc1 & Nuc2
SEQ ID NO:6: Forward primer for GAPDH
SEQ ID NO:7: Reverse primer for GAPDH
SEQ ID NO:8: Forward primer for NTS
SEQ ID NO:9: Reverse primer for NTS
SEQ ID NO:10: Forward primer for IER5L
SEQ ID NO:11: Reverse primer for IER5L

What is claimed is:

1. A conjugate comprising a pyrrole-imidazole polyamide that recognizes a specific DNA sequence and a bromodomain inhibitor.

2. The conjugate according to claim 1, wherein the bromodomain inhibitor is a compound that interacts with a bromodomain contained in a protein selected from the group consisting of a histone acetyltransferase, a histone methyltransferase, an ATP-dependent chromatin-remodeling complex protein, and a BET protein.

3. The conjugate according to claim 2, wherein the bromodomain inhibitor is a compound that interacts with a histone acetyltransferase.

4. The conjugate according to claim 1, wherein the bromodomain inhibitor is a 5-isoxazolyl-benzimidazole compound.

5. The conjugate according to claim 4, wherein the 5-isoxazolyl-benzimidazole compound is a compound represented by the following formula: 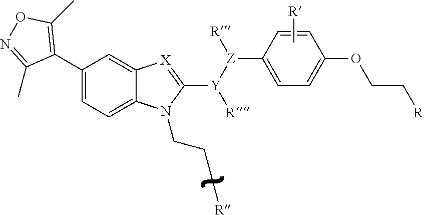
wherein,
R is NH₂ or NHCOCH₂CH₂CH₂COOH,
R′ is H, CH₃, F, Cl, or NO₂,
R″ is a group represented by the following formula: 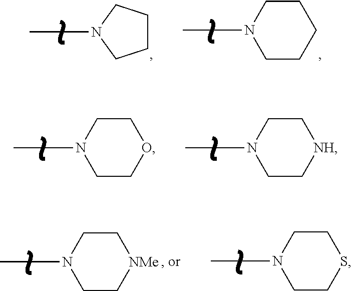
R′″ is H or CH₃,
R″″ is H or CH₃,
X is CH or N,
Y is CH or O, and
Z is CH or O.

6. The conjugate according to claim 3, which is represented by Formula I: 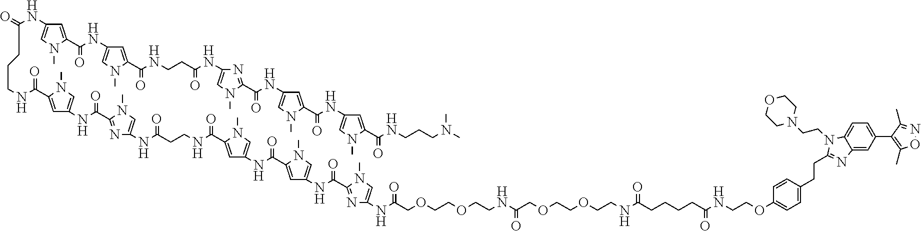 [SMILES: Cc1noc(C)c1-c1ccc2c(c1)nc(CCc1ccc(OCCNC(=O)CCCCC(=O)NCCOCCOCC(=O)NCCOCCOCC(=O)Nc3cn(C)c(C(=O)Nc4cc(C(=O)Nc5cc(C(=O)NCCC(=O)Nc6cn(C)c(C(=O)Nc7cc(C(=O)NCCCC(=O)Nc8cc(C(=O)Nc9cc(C(=O)NCCC(=O)Nc%10cn(C)c(C(=O)Nc%11cc(C(=O)Nc%12cc(C(=O)NCCCN(C)C)n(C)c%12)n(C)c%11)n%10)n(C)c9)n(C)c8)n(C)c7)n6)n(C)c5)n(C)c4)n3)cc1)n2CCN1CCOCC1]
or Formula II:

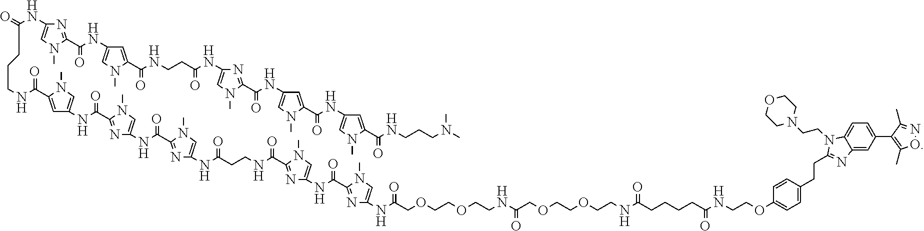 [SMILES: Cc1noc(C)c1-c1ccc2c(c1)nc(CCc1ccc(OCCNC(=O)CCCCC(=O)NCCOCCOCC(=O)NCCOCCOCC(=O)Nc3cn(C)c(C(=O)Nc4cn(C)c(C(=O)NCCC(=O)Nc5cn(C)c(C(=O)Nc6cn(C)c(C(=O)Nc7cc(C(=O)NCCCC(=O)Nc8cn(C)c(C(=O)Nc9cc(C(=O)NCCC(=O)Nc%10cn(C)c(C(=O)Nc%11cc(C(=O)Nc%12cc(C(=O)NCCCN(C)C)n(C)c%12)n(C)c%11)n%10)n(C)c9)n8)n(C)c7)n6)n5)n4)n3)cc1)n2CCN1CCOCC1]

7. A composition for regulating biochemical activity, comprising the conjugate according to claim 1.

8. A composition for regulating modification of histone, comprising the conjugate according to claim 1.

9. A method of DNA sequence-selectively acetylating a nucleosome, the method comprising bringing the conjugate according to claim 3 into contact with a sample containing a nucleosome.

10. A composition for recruiting a bromodomain-containing protein, comprising the conjugate according to claim 1.

11. A method of DNA sequence-selectively recruiting a bromodomain-containing protein, the method comprising bringing the conjugate according to claim 1 into contact with a sample.


SEQUENCE LISTING (11 sequences)

SEQ ID NO: 1 - DNA, 223 bp, Artificial
  Template DNA
tctccgactcagaacagtcagggaacagtcagggaacagtcagggaacagtcagggaacagtcaggggcggccgccctggagaatcccggtgccgaggccgctcaattggtcgtagacagctctagcaccgcttaaacgcacgtacgcgctgtcccccgcgttttaaccgccaaggggattactccctagtctccaggcacgtgtcagatatatacatcctgt

SEQ ID NO: 2 - DNA, 223 bp, Artificial
  Template DNA
tctccgactcagagccgccatttagccgccatttagccgccatttagccgccatttagccgccatttgcggccgccctggagaatcccggtgccgaggccgctcaattggtcgtagacagctctagcaccgcttaaacgcacgtacgcgctgtcccccgcgttttaaccgccaaggggattactccctagtctccaggcacgtgtcagatatatacatcctgt

SEQ ID NO: 3 - DNA, 19 bp, Artificial
  Primer
tctccgactcagaacagtc

SEQ ID NO: 4 - DNA, 18 bp, Artificial
  Primer
tctccgactcagagccgc

SEQ ID NO: 5 - DNA, 17 bp, Artificial
  Primer
gcaccgggattctccag

SEQ ID NO: 6 - DNA, 20 bp, Artificial
  Primer
accacagtccatgccatcac

SEQ ID NO: 7 - DNA, 20 bp, Artificial
  Primer
tccaccaccctgttgctgta

SEQ ID NO: 8 - DNA, 23 bp, Artificial
  Primer
tgaccaatatgcatcatcaaaga

SEQ ID NO: 9 - DNA, 21 bp, Artificial
  Primer
tcttgcaacaagctcctcttc

SEQ ID NO: 10 - DNA, 20 bp, Artificial
  Primer
aagatccacagctcccgaac

SEQ ID NO: 11 - DNA, 19 bp, Artificial
  Primer
gtagcgctcgctcaggtag